(19) United States
(12) Patent Application Publication
(10) Pub. No.: US 2009/0192298 A1
(43) Pub. Date: Jul. 30, 2009

(54) Through-bond energy transfer cassettes, systems and methods

(76) Inventor: Kevin Burgess, College Station, TX (US)

Correspondence Address: BAKER BOTTS L.L.P., 2001 ROSS AVENUE, SUITE 600, DALLAS, TX, 75201-2980, US

(21) Appl. No.: 12/291,785

(22) Filed: Nov. 13, 2008

Related U.S. Application Data
(60) Provisional application No. 60/987,569, filed on Nov. 13, 2007.
(60) Provisional application No. 60/988,347, filed on Nov. 15, 2007.

(51) Int. Cl. C07K 14/00; C07F 5/02; C07D 311/78; C07D 265/34; C07H 21/00

(52) U.S. Cl. 530/402; 548/405; 548/110; 549/394; 544/99; 536/22.1; 536/1.11

(57) ABSTRACT

The present disclosure relates, according to some embodiments, to compositions, systems, and methods for preparing and using fluorescent through-bond energy transfer cassettes.

### CROSS-REFERENCE TO RELATED APPLICATIONS

This application claims the benefit of U.S. Provisional Patent Application Ser. No. 60/987,569, filed Nov. 13, 2007, and claims the benefit of U.S. Provisional Patent Application Ser. No. 60/988,347, filed Nov. 15, 2007, the contents of which are hereby incorporated in their entirety by reference.

### FIELD OF THE DISCLOSURE

The present disclosure relates, in some embodiments, compositions, systems, and/or methods for labeling and/or detecting a molecule. For example, a composition may include a dye compound (e.g., a fluorescent dye compound) that supports through-bond energy transfer.

### BACKGROUND OF THE DISCLOSURE

Fluorescent tags may be used in a variety of applications including, for example, biological experiments. In such experiments, a general approach may be to tag each biological molecule with a fluorescent group, then excite the group at one wavelength. For instance, an argon laser may be used to excite in the 488/512 nm range. This approach may require several molecules to be labeled with distinct fluorescent tags, commonly referred to as multiplexing.

However, this technique suffers from a serious defect. Dyes that emit at longer wavelengths absorb at the excitation wavelength less efficiently, thereby diminishing their fluorescence intensities. This may be especially problematic when one wishes to detect low levels of fluorescence or sensitivity.

One solution to this problem has been to use fluorescence resonance energy transfer (FRET) between two dyes, a donor and an acceptor. However, the resolution of emissions from multiplexed FRET-based cassettes is constrained by the requirement that the fluorescence of the donor overlap with the absorption of the acceptor. (The overlap must correspond to the overlap integral in Förster energy transfer.)

### SUMMARY

Accordingly, a need has arisen for improved fluorescent through-bond energy transfer cassettes. The need for cassettes useful for multiplexing in molecular biology, biochemistry and biotechnology may be particularly acute.

The present disclosure relates, according to some embodiments, to compositions, systems, and methods for preparing and using fluorescent through-bond energy transfer cassettes. For example, the present disclosure includes a cassette comprising of a donor component including a fluorescein and an acceptor component capable of fluorescing. These components may be linked by a linker, which connects the donor and acceptor components in a non-planar conformation. A linker may electronically conjugate donor and acceptor components to allow through-bond energy transfer.

In some embodiments of the present disclosure, an acceptor component may include rhodamine, an extended fluorescein, and/or derivatives thereof. A linker may include one or more double bonds. A functional group, such as a carboxyl group, may be added to a cassette to facilitate coupling of the cassette with a biological molecule (e.g., a protein, a nucleic acid, a lipid, a carbohydrate, and/or combinations thereof).

In some embodiments, cassettes in a system may be selected so that fluorescence from each cassette may be distinguished from fluorescence from every other cassette. A cassette may also each have a molecular weight that varies by no more than 10% from that of any other cassette of a system. A system may be used to label a pool of biological molecules through multiplexing. These biological molecules may be nucleotides.

A system may include at least two fluorescent through-bond energy transfer cassettes as described above, in some embodiments. In some embodiments, a system may include at least 4 cassettes, each of which is attached to a different nucleotide in a DNA molecule. Such a system may be used in DNA sequencing.

Cassettes, in some embodiments, may be prepared by a reaction with a microwave heat source. A heat source may be used in the linking reaction. Acceptor components may also be prepared using such a heat source.

Some embodiments of the disclosure relate to a selected from the group consisting of dye 1, dye 2, dye 3, dye 4, dye 5, dye 6, dye 9, dye 10, dye 11, dye 12, dye 13, dye 14, dye 15, dye 16, dye 17, dye 18, dye 19, and dye 20. Dye 6, for example, may be represented by the structure shown in FIG. 3. R¹ may comprise an alkyl, a cycloalkenyl, an iodoalkene, an alkenyl ester, and/or an nitroalkene, non-limiting examples of which are provided in FIG. 3. R² may comprise H and/or alkyl, non-limiting examples of which are provided in FIG. 3. R³ may comprise H and/or sulfate, non-limiting examples of which are provided in FIG. 3. In some embodiments, a dye may comprise dye 9, wherein “Nu” may comprise a nucleophile and/or a nucleophilic amine and/or NR₂ may comprise an amine, a cycloamine, and/or a cyclo secondary amine, non-limiting examples of which are provided in Table 2.

The present disclosure relates to a method for sulfonating (e.g., monosulfonating) a dye (e.g., a sulfonated 4,4-difluoro-4-bora-3a,4a-diaza-s-indacene). For example, a method may comprise contacting an acid (e.g., chlorosulfonic acid) and a sulfonated 4,4-difluoro-4-bora-3a,4a-diaza-s-indacene in an organic solvent (e.g., CH₂Cl₂) at a sub-zero temperature (e.g., from about −45° C. to about −20° C.) to form a mixture In some embodiments, a method may comprise warming (e.g., slowly warming) the mixture to about room temperature (e.g., about 20° C.) with or without the addition of heat. The rate of warming may be adjusted, for example, by using a bath (e.g., a water bath) to slow or accelerate warming relative to the warming that would occur if simply exposed to room air under ambient conditions. A method may comprise, according to some embodiments, contacting (e.g., quenching) the mixture with a base (e.g., bicarbonate).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the disclosure may be understood by referring, in part, to the present disclosure and the accompanying drawings, wherein:

FIG. 1 illustrates some specific examples of fluorescent dyes;

FIG. 2 illustrates some fluorescent dyes according to specific example embodiments of the disclosure;

FIG. 3 illustrates some fluorescent dyes according to specific example embodiments of the disclosure;

FIG. 4 illustrates a scheme for preparing some fluorescent dyes according to specific example embodiments of the disclosure;

FIG. 5 illustrates a scheme for preparing some fluorescent dyes according to specific example embodiments of the disclosure;

FIG. 6 illustrates some fluorescent dyes according to specific example embodiments of the disclosure;

FIG. 7 illustrates some fluorescent dyes according to specific example embodiments of the disclosure;

FIG. 8 illustrates some specific examples of fluorescent dyes;

FIG. 9 illustrates some fluorescent dyes according to specific example embodiments of the disclosure;

FIG. 10 illustrates some fluorescent dyes according to specific example embodiments of the disclosure;

FIG. 11 illustrates some fluorescent dyes according to specific example embodiments of the disclosure;

FIG. 12A illustrates some energy transfer cassettes according to specific example embodiments of the disclosure;

FIG. 12B illustrates an energy transfer cassette 33 according to specific example embodiments of the disclosure;

FIG. 13 illustrates some energy transfer cassettes according to specific example embodiments of the disclosure;

FIG. 14 illustrates some energy transfer cassettes according to specific example embodiments of the disclosure;

FIG. 15 illustrates some energy transfer cassettes according to specific example embodiments of the disclosure; and

FIG. 16 illustrates some energy transfer cassettes according to specific example embodiments of the disclosure.

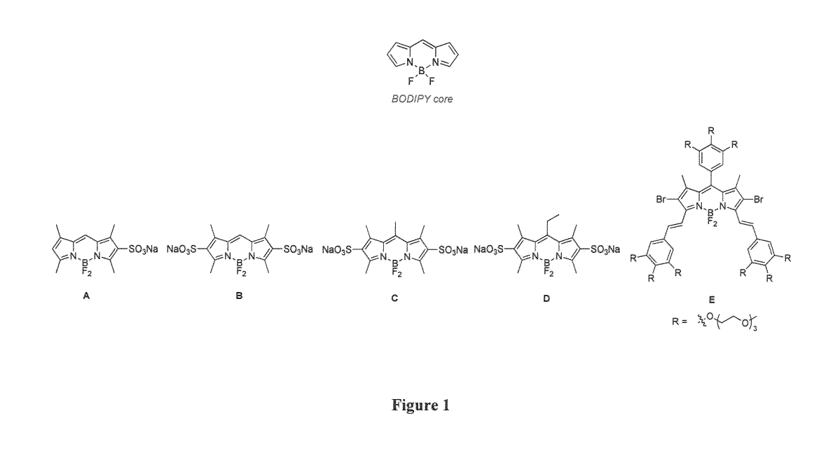

### DETAILED DESCRIPTION

In biotechnology, FRET-based systems may be efficiently replaced with cassettes that have donors connected to acceptors via electronically conjugated linkers. Energy transfer in such systems may occur through bonds and/or through space. Through-bond energy transfer systems tend to be fast relative to non-radioactive decay pathways used by FRET, so there is no obvious upper limit to the separation between the wavelengths at which the donor absorbs energy and the acceptor emits it. Accordingly, multiplexing with through-bond energy transfer cassettes does not necessarily involve loss of sensitivity at high resolutions.

The present disclosure relates, in some embodiments, to a fluorophore (e.g., a sulfonated 4,4-difluoro-4-bora-3a,4a-diaza-s-indacene (BODIPY) dye) and/or a method for sulfonating a fluorophore (e.g., a BODIPY dye).

The present disclosure relates, according to some embodiments, to fluorescent through-bond energy transfer cassettes. Donor and acceptor fragments may be connected by linkers with extended π-systems, or may be directly connected (no linkers), such that electronic conjugation between the donor and acceptor systems could occur if they were made planar. If the two compounds maintain discrete donor and acceptor components (i.e., the π-systems do not simply merge to form one extended, conjugated system) then they may be called through-bond energy transfer cassettes. This name should not be taken to mean that through-space energy-transfer cannot occur as well. It simply implies that direct transfer of energy between the donor and acceptor π-systems can occur simultaneously through bonds.

Such cassettes may be used for a variety of purposes, including attachment to biological molecules (e.g., proteins, nucleic acids, carbohydrates, lipids, and/or combinations thereof). Multiple cassettes may be used in combination with one another or other compounds for multiplexing and resolution of different molecules within the same system. For example, a system for use in multiplexing may comprise a plurality of acceptors that each emit over a narrow range of wavelengths, each of which is distinct from the others, thus facilitating differentiation of the cassettes' signals.

Cassettes may include a donor component with strong absorbance at its excitation wavelength, an acceptor component that fluoresces with high quantum yield in an aqueous environment, and a suitable conjugated linker. A cassette, in some embodiments, may include functional groups to allow attachment of the cassette to a biological molecule. Non-limiting examples of a functional group include activated carboxilic acids (e.g., to bind lysine on proteins and/or amino functionalized nucleic acids); bromoacyl groups for binding thiols (e.g., cytokine in proteins); amino groups which may be modified to isothiocyanate (e.g., to allow binding to lysines in a protein and/or amino-modified nucleic acids); and/or complimentary azide or alkyne groups (e.g., to cycloadd, azide or alkynes groups on a protein or DNA). A donor, acceptor, and/or linker may independently comprise a functional group.

A donor and/or an acceptor may include a fluorophore. For example, a donor may include a fluorescein molecule and the acceptor may includes a rhodamine-like component or an extended fluorescein molecule that may be varied to obtain a set of dyes with desired emission characteristics.

A linker may be selected so as to prevent the donor and acceptor components from having a planar arrangement because such an arrangement would result in the system behaving as a single conjugated dye. However, the linker also preferably allows through-bond energy transfer from the donor to the acceptor that is rapid relative to non-radioactive decay processes such as FRET. In some embodiments, the linker is an electronically conjugative double bond.

According to some embodiments, electronic conjugation between the donor and acceptor π-systems of a through-bond energy cassette (which are each planar) could occur if they are made co-planar with each other. A cassette may be configured and arranged such that donor and acceptor π-systems are sterically hindered (e.g., prevented) from becoming co-planar in the ground state. This steric hinderence may be introduced by a rotation (“twist”) between two aromatic fragments in the cassette. Upon excitation (e.g., to a non-ground state), donor and acceptor π-systems may become co-planar such that electronic conjugation may occur.

A cassette (e.g., a through-bond energy cassette) may have, in some embodiments, (i) one or more donor entities for harvesting exciting photons, (ii) one or more acceptor entities for emitting photons as fluorescence, (iii) a twisted but otherwise conjugated shape that prevents the donor and acceptor parts from electronically coupling into a single planar dye system, and/or (iv) a functional group or other special physical property that allows them to be attached or associated with dye molecules. In some embodiments, a cassette (e.g., a through-bond energy cassette) may have (a) solubility in aqueous media, (b) a high cross-section for UV absorbance by the donor at a useful, excitation wavelength, (c) efficient energy transfer from the donor to the acceptor, (d) sharp fluorescence emissions with high quantum yields from the acceptor part, and/or (e) high quantum yield for the whole cassette, i.e., (photons emitted as fluorescence from the acceptor)/(photons absorbed in the donor absorption region) should be high.

Through bond energy transfer cassettes may include, for example, combinations of many dyes fragments. According to some embodiments, fragments may include BODIPY, azaBODIPY, xanthene (fluoresceines and derivatives), pyroniin (rhodamines and derivatives), and benzophenoxazines. Other dye fragments that may be used include those derived from squaraines, cyanines and coumarins.

In some embodiments, a cassette may be used in a variety of applications. For example, proteins, polypeptides, and amino acids may be labeled for proteomics, sequencing, imaging and other biochemical applications. Nucleic acids such as DNA or RNA or nucleotides may be labeled for genomics, high-throughput sequencing, and genetyping applications. According to some embodiments, synthetic or non-naturally occurring nucleotides and amino acids may be labeled or included in labeled molecules.

A cassette may also be put to other uses, such as dye coloring of clothes or cosmetic products (e.g., hair or skin dyes or pigments). One or more cassettes may be incorporated into materials with unusual fluorescence or optical properties.

Sets of energy transfer dyes may be used for applications in which several molecular entities are to be excited using the same wavelength (corresponding to the same donor in each cassette), but emit at different wavelengths (different acceptors), i.e., multiplexing. An example of this type of application are in DNA sequencing wherein one label is used in each of the four sequencing reactions. The excitation wavelength used in most contemporary DNA sequencing machines is the same, but the fluorescence emissions should be sharp, well resolved, and distinct for each label used in each experiment. Another example featuring multiplexing would be to use several TBET cassettes with the same donor to label several different proteins in a cell, excite them all simultaneously using a single wavelength excitation source, and observe distinct emission wavelengths characteristic of the labels used on each protein. In a simple example, two proteins may be labeled (i.e., may be each bound to a separate cassette) such that, while sharing a common donor, each has a unique acceptor. Co-localization may be assessed by exposing the proteins to the excitation wavelength and monitoring the emissions from the respective acceptors.

### Dye Types 1-2: BODIPY Dyes

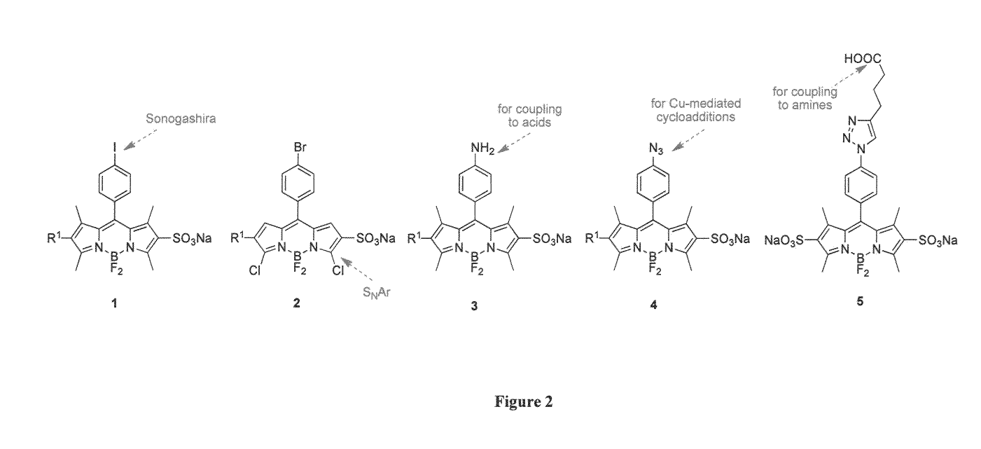

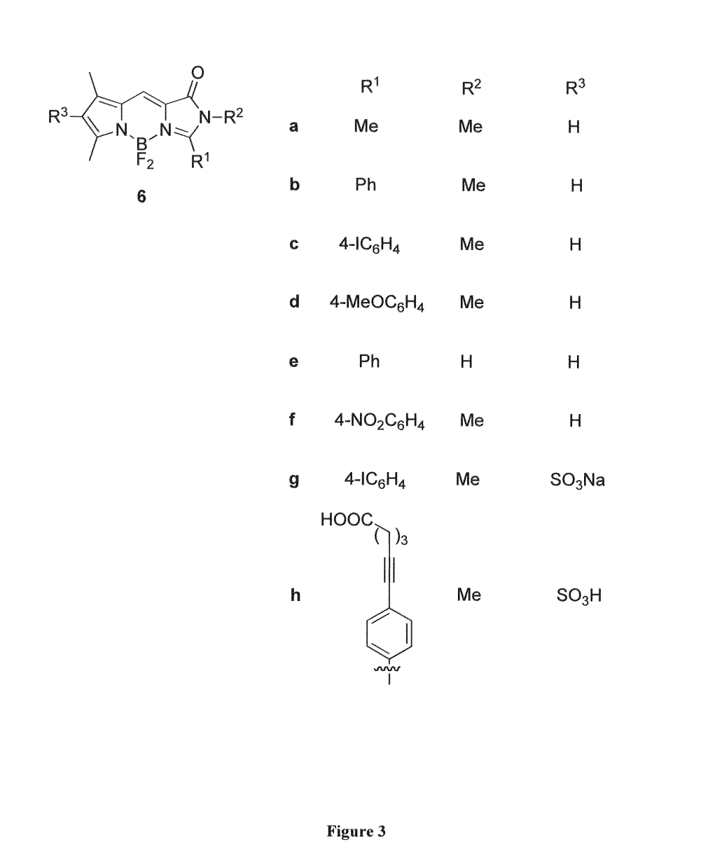

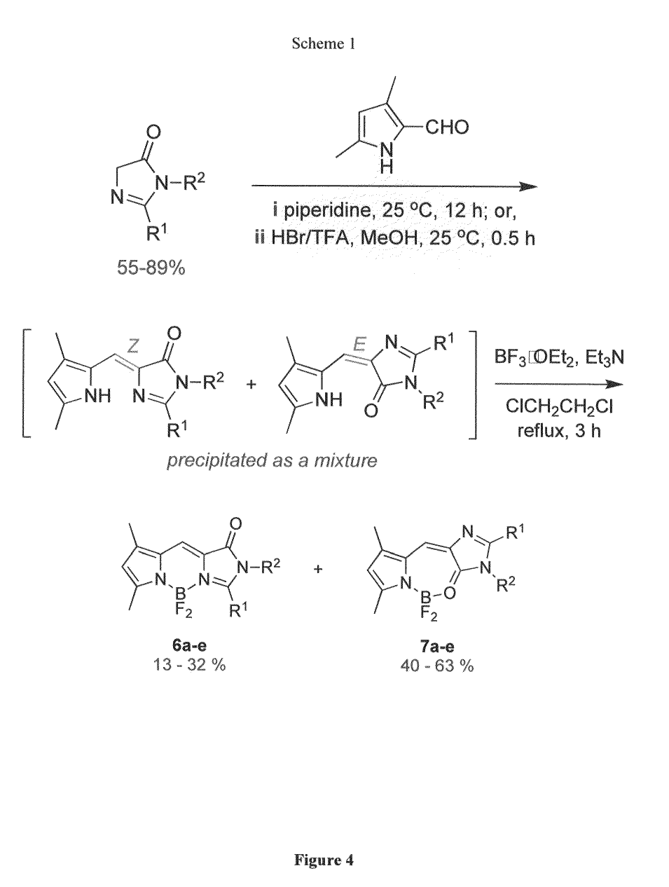

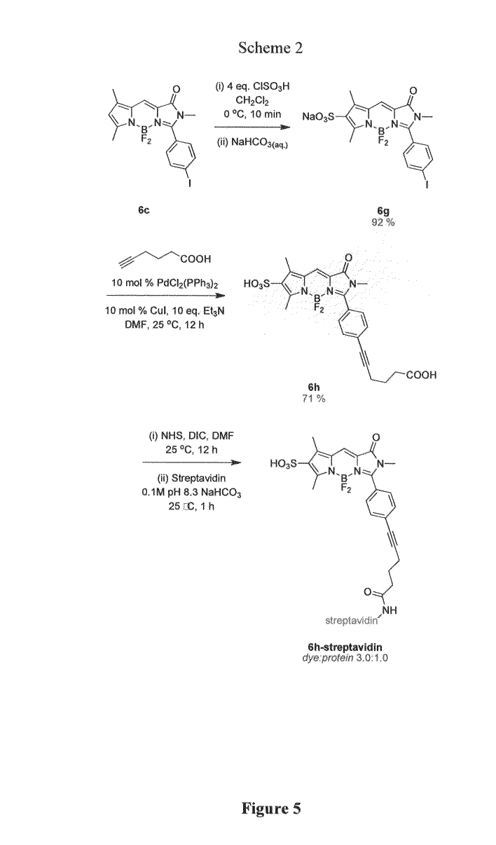

In some embodiments a fluorophore may comprise a BODIPY (e.g., a sulfated BODIPY dye and/or a water soluble BODIPY dye). A sulfonated BODIPY dye may comprise, for example, a dye shown in FIG. 2 (structures 1-5), a dye shown in FIG. 3 (structure 6), and/or variations thereof. An example embodiment of a method for preparing a sulfonated BODIPY dye is shown in FIG. 2 and is presented in Example 1. An example embodiment of a method for preparing a sulfonated BODIPY dye is shown in FIG. 3. In some embodiments, a method for synthesizing a sulfonated BODIPY may comprise Scheme 1 (FIG. 4). A modification to obtain compound 6h is shown in Scheme 2 (FIG. 5), along with an illustrative procedure for coupling this dye to a protein (compound 7 may be regarded as a byproduct).

A fluorophore, according to some embodiments of the disclosure, may have a high extinction coefficient and/or a sharp, intense fluorescence emission (e.g., rivaling that of the BODIPY dyes). A fluorophore may also have single molecule detection capacity. Table 1 gives some illustrative data.

TABLE 1

| | I_{max abs} | e | I_{max emiss} | fwhm | | |
|---|---|---|---|---|---|---|
| | (nm) | (M⁻¹cm⁻¹) | (nm) | (nm) | F^{a} | solvent |
| 6a | 477 | 57286 | 485 | 19 | 0.89 ± 0.01 | MeOH |
| 6b | 490 | 58684 | 521 | 46 | 0.87 ± 0.01 | MeOH |
| 6c | 492 | 57303 | 532 | 54 | 0.86 ± 0.02 | MeOH |
| 6d | 493 | 51269 | 521 | 38 | 0.85 ± 0.03 | MeOH |
| 6e | 495 | 37867 | 531 | 48 | 0.80 ± 0.01 | MeOH |
| 6f | 492 | 44003 | 598 | 120 | 0.0004 | MeOH |
| 6f | 498 | — | 587 | 53 | 0.53 ± 0.01 | Hexanes |
| 6g | 488 | 48132 | 531 | 53 | 0.88 ± 0.01 | MeOH |
| 6g | 481 | 46461 | 518 | 50 | 0.87 ± 0.01 | Phos 7.4^{b} |
| 6h | 488 | 35024 | 538 | 58 | 0.84 ± 0.01 | MeOH |
| 6h | 482 | 34766 | 526 | 56 | 0.82 ± 0.01 | Phos 7.4^{b} |
| 6h- | 482 | — | 529 | 56 | — | Phos 7.4^{b} |
| strep | | | | | | |

^{a}Fluorescein in 0.1M NaOH as standard (φ = 0.92);
^{b}0.1M lithium phosphate buffer (pH = 7.4)

### Dye Type 3: Systems Related to Rosamines and Rhodamines

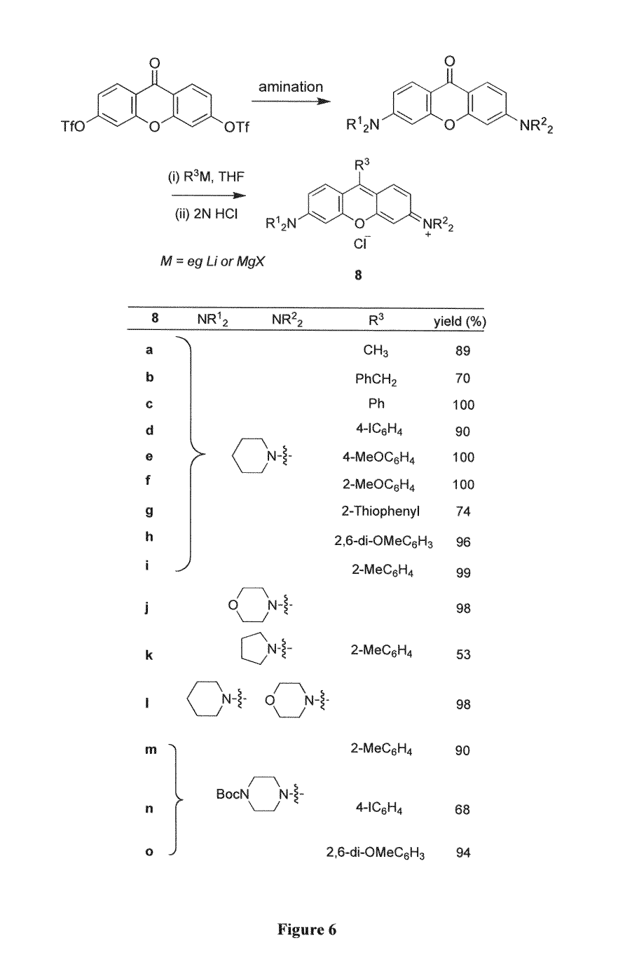

In some embodiments a fluorophore may comprise a rosamine (e.g., a rhodamine less one carboxylic acid). A rosamine may comprise one or more of the compounds shown in FIG. 6 and/or variations thereof. For example, the central aryl group on any of the compounds shown may be replaced by a heteroatom.

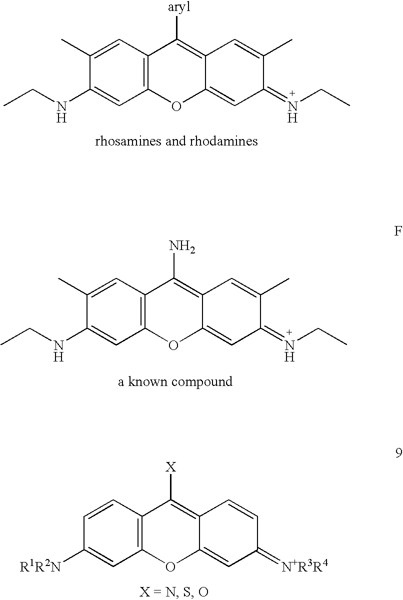

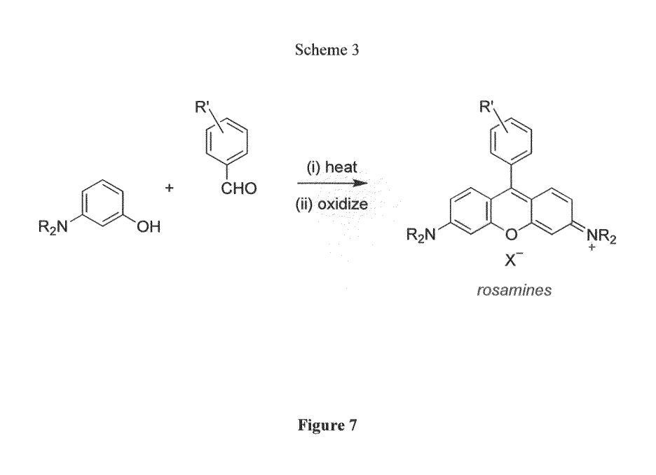

A rosamine may be prepared, in some embodiments, according to Scheme 3 (FIG. 7). The amination step may be via direct nucleophilic attack, or via metal-complex catalyzed amination methods. This route may be desirable because it allows diverse functionalities to be introduced into the dyes, so affording materials that are useful for labeling biomolecules, and for the production of through bond energy transfer cassettes.

A dye (e.g., a rosamine dye) having structure 9 may be prepared, according to some embodiments, as shown in Table 2. In some embodiments, “Nu” may comprise a nucleophile and/or a nucleophilic amine and/or NR₂ may comprise an amine, a secondary amine and/or a cyclo secondary amine.

TABLE 2

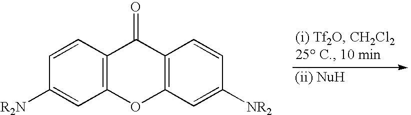

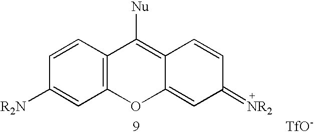

| 2 | NR₂ | NuH | yield (%) |
|---|---|---|---|
| a | | 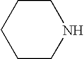 | 81 |
| b | | Et₂NH | 61 |
| c | 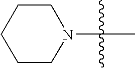 | rac-Sec-BuNH₂ | 98 |
| d | | 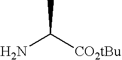 | 80 |
| e | | BnSH | 23 |
| f | 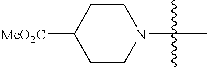 | 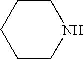 | 86 |

In some embodiments, diverse functionalities may be introduced into the dyes using the method of Table 2 or the like, so affording materials that are useful for labeling biomolecules, and for the production of through bond energy transfer cassettes.

Spectral properties for some specific example dyes are shown in Table 3.

TABLE 3
Spectral Properties of 9 in CH₂Cl₂

| | Iₘₐₓ | | I_{f} | fwhm | |
|---|---|---|---|---|---|
| 9 | (nm) | eₘₐₓ | (nm) | (nm)^{a} | F_{f}^{b} |
| a | 491 | 60409 | 562 | 55 | 0.14 ± 0.01 |
| b | 499 | 50978 | 558 | 41 | 0.11 ± 0.02 |
| c | 456 | 52423 | 537 | 52 | 0.85 ± 0.01 |
| d | 462 | 62044 | 544 | 52 | 0.90 ± 0.02 |
| e | 598 | 82617 | 619 | 38 | 0.79 ± 0.02 |
| f | 482 | 54908 | 552 | 47 | 0.02 ± 0.001 |

^{a}Full width at half-maximum height: a measurement of the sharpness of the fluorescence peaks;
^{b}Rhodamine 101 in EtOH (Φ = 1.0) was use as the standard.

### Dye Type 4: Functionalized Dichlorofluorescein Dyes

Fluorescein dyes may undergo reactions in response to pH changes, e.g. protonation of phenolic groups and cyclization to lactone forms. These changes may tend to occur around physiological pH values, and this complicates interpretation of their fluorescence properties. In response, fluorine substitutents may be placed on the fluorescein nucleus, making the phenol groups less pH sensitive under physiological conditions, and reducing pH/fluorescence interdependence. The photostability properties may also be improved.

Incorporation of fluorine into fluorescein-dyes can be experimentally difficult and may involve expensive reagents. Thus, according to some embodiments, a chlorinated-fluorescein may be used to achieve the same advantages as the fluorinated compounds. For example, compounds 10-12 have substituents that may be highly desirable. Substances 10-12 are particularly valuable for the preparation of through-bond energy transfer cassettes, while 12 is also useful for labeling biomolecules via copper mediated azide-alkyne cycloadditions.

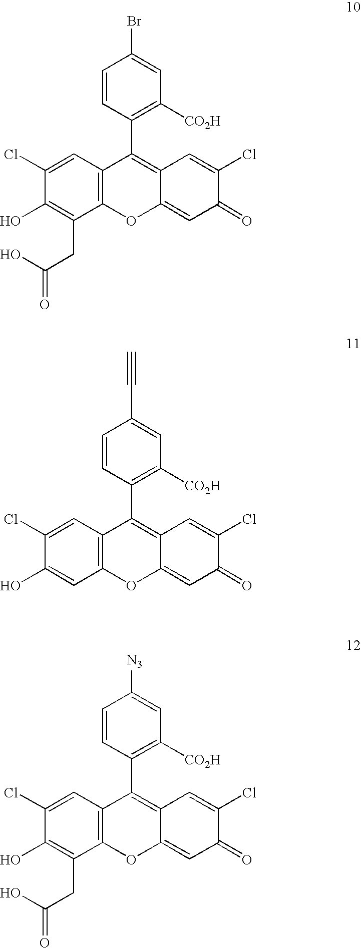

### Dye Type 5: Functionalized Dichlorofluorescein Dyes

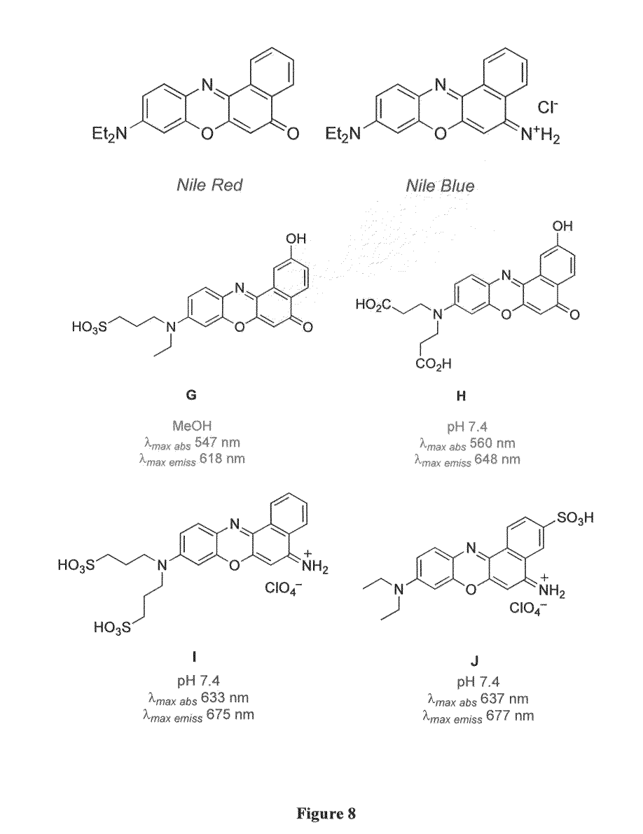

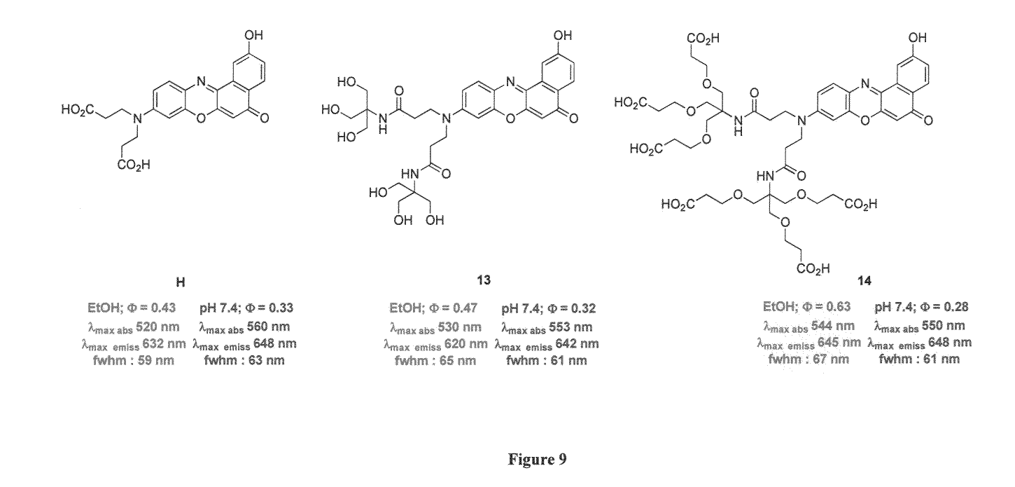

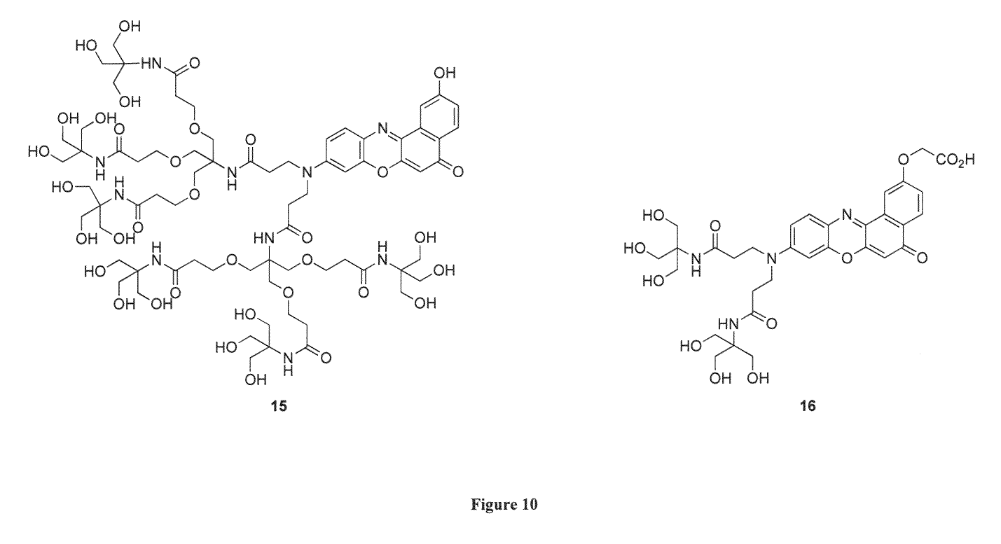

Under some conditions Nile Red and Nile Blue may have poor water solubility. Variations that may display desirable water solubility may include compounds G-J (FIG. 8). According to some embodiments of the disclosure, a fluorophore may include dye 13 and/or dye 14. Dyes 13 and/or 14 may be water soluble. Without limiting any embodiment the disclosure to any particular theory or mechanism of action, dyes 13 and 14 may illustrate the concept of shielding a hydrophobic benzophenoxazine dye from an aqueous environment by surrounding it with “dendric-wedge” molecules. Dendric shielding may (i) increase the overall water solubility of a system; (ii) encapsulate the hydrophobic core in a medium that is less polar than water (which may enhance the dye's fluorescence properties); and, (iii) provide the potential to introduce different functional groups on the surface of the molecules that may be useful for conjugating them to biomaterials. Phenolic function of dyes like 13 and 14 may be converted into triflates, which may be suitably functionalized for incorporation into through-bond energy transfer cassettes.

A dye (e.g., dye 16) may be used, according to some embodiments, in a “SNAP-tag” method, wherein a dye may be coupled to a nucleobase derivative (e.g. guanidine) for transfer to an enzyme like O⁶-alkylguanidine DNA transferase (AGT); this methodology may be used to label AGT-conjugates with other proteins. Without limiting any embodiment of the disclosure to any particular mechanism of action or theory, efficacy of these methods may relate to the functionality and/or water solubility of dyes.

A dye may include, in some embodiments, one or more of Nile Blue derivatives 17-20 (FIG. 11). These have fluorescence properties that may be superior to any water-soluble Nile Blue derivative that has been reported before as gauged by at least one of the following metrics: (i) fluorescence quantum yield in aqueous media; (ii) the presence of a functional group that enables them to be coupled to biomolecules or incorporated into through-bond energy transfer cassettes; (iii) the sharpness of the fluorescence emission in aqueous media; (iv) long wavelength fluorescence emission maxima in aqueous media; and/or, (v) high water solubility aqueous media. For example, the advantages of water soluble Nile Blue derivatives 17 and 18 over I and J may include: (i) sharper fluorescence peaks (fwmh 20 nm less than reported water-soluble Nile Blue derivatives in phosphate buffer pH 7.4); (ii) improved quantum yields at pH 7.4 in aqueous media; (iii) condensation reactions to form 17 and 18 require no acid or harsh reaction conditions (reactions were performed in DMF at 90° C. without any acids or bases; (iv) purification was done using reverse phase medium pressure liquid chromatography (MPLC) using acetonitrile and water as eluting solvents; (v) functional groups in 17 and 18 may be further derivatized for covalently attaching the dye molecules to proteins or for incorporation into through bond energy transfer cassettes; (vi) the dyes fluoresce in the near IR region (670-680 nm) in aqueous media.

In some embodiments, compounds 17-20 and similar materials may be functionalized with dendric wedge groups as described above, and/or with other groups that enable them to be used in SNAP tag methods.

### Through Bond Energy Transfer Cassettes

According to some embodiments, a through-bond energy transfer cassette may include compounds with simple aromatic groups as donors and BODIPY systems as acceptors. For example, a through-bond energy cassette may include xanthene and pyronin (i.e. fluorescein- and rhodamine-like cassettes). These have desirable donor extinction coefficients, but may lack desirable water solubility.

A cassette, in some embodiments, may include a BODIPY dye donor, a BODIPY dye acceptor, an AzaBODIPY donor, and/or an AzaBODIPY acceptor. According to some embodiments, donor and acceptor parts may be electronically conjugated via a π-bond system were it not for the fact that they are prevented from becoming co-planar. Donor molecules harvest radiation and transmit it to the acceptor part via mechanisms that involve significant transfer of the energy through that π-bond system.

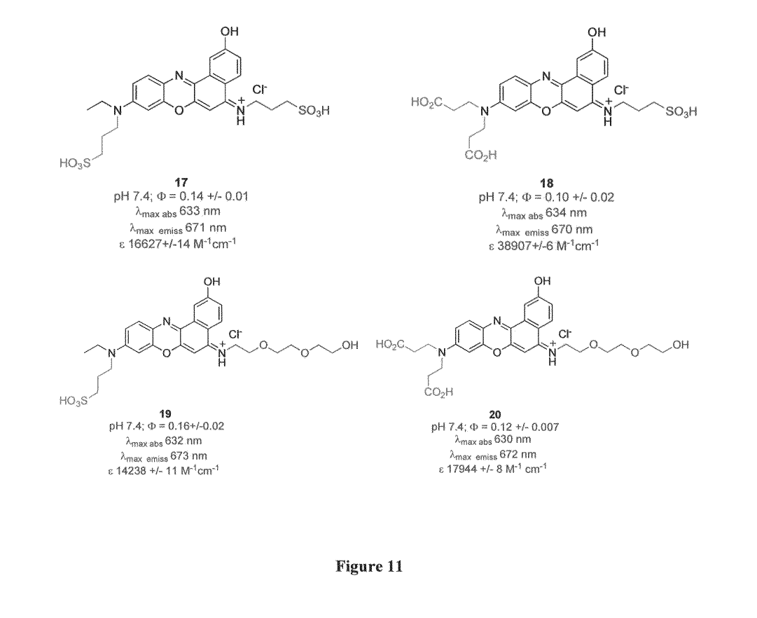

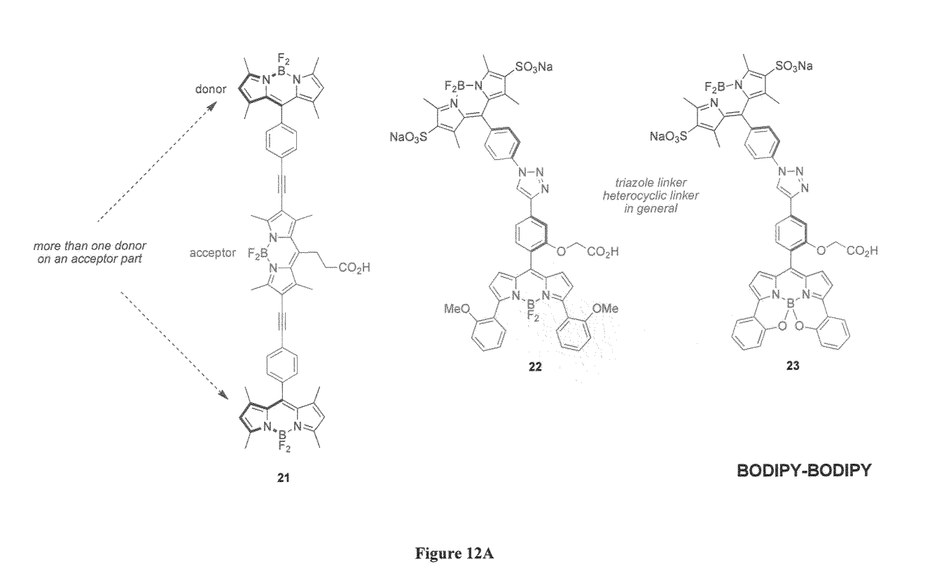

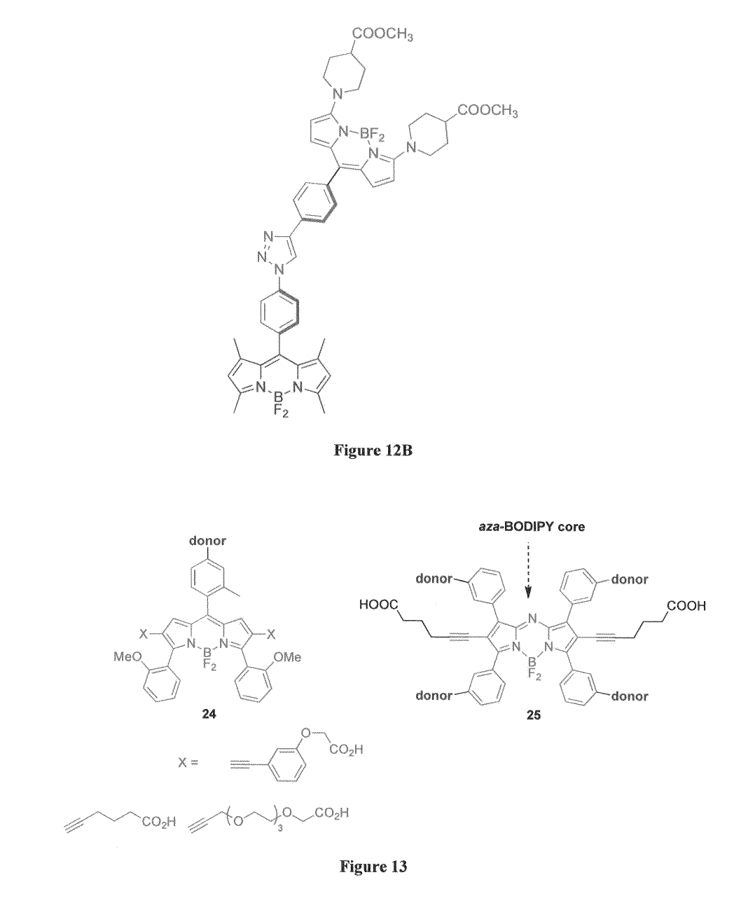

Cassettes 21-22 (FIG. 12A) are an example of a BODIPY-BODIPY system. Compound 21 comprises two donor BODIPY parts (shown in blue) directly attached to the red part that may be considered as one acceptor fragment since the —C₆H₄CC— part can become co-planar with the red BODIPY core. However, the two blue BODIPY parts cannot become coplanar with the rest of the molecule for steric reasons; this is less conjugated overall than the red part, absorbs at shorter wavelengths (higher energy) and relays the energy absorbed through bonds to the red part. This accepts the energy, and emits it at a longer wavelength. This cassette may not transmit energy via a F6rster mechanism (fluorescence resonance energy transfer) because the orientation factor excludes this. So, any energy transfer observed for this cassette has to come via some other mechanism. The carboxylic acid functionality on the red part allows this cassette to be conjugated to functionalities (usually amines) on biomolecules. This particular cassette may not be water soluble.

Cassette 22 however, is water soluble. It has a disulfonated donor part (blue) that cannot come into conjugation with the central region colored in purple. That purple region also cannot become conjugated to the red acceptor part (another BODIPY that emits at longer wavelengths), so that purple part might be considered as a linker. Cassette 22 may have particularly desirable energy transfer properties, and be conjugated to biomolecules. In some embodiments, a BODIPY-BODIPY cassette may include a triazole linker.

The (red) acceptor part of cassette 23 emits at an even longer wavelength than the corresponding fragment in 22, so the difference between the absorption maximum for the donor and the fluorescence maximum of the acceptor (this might be called a “pseudo-Stokes' shift”) may be greater. A cassette may also include the cassette shown in FIG. 12B.

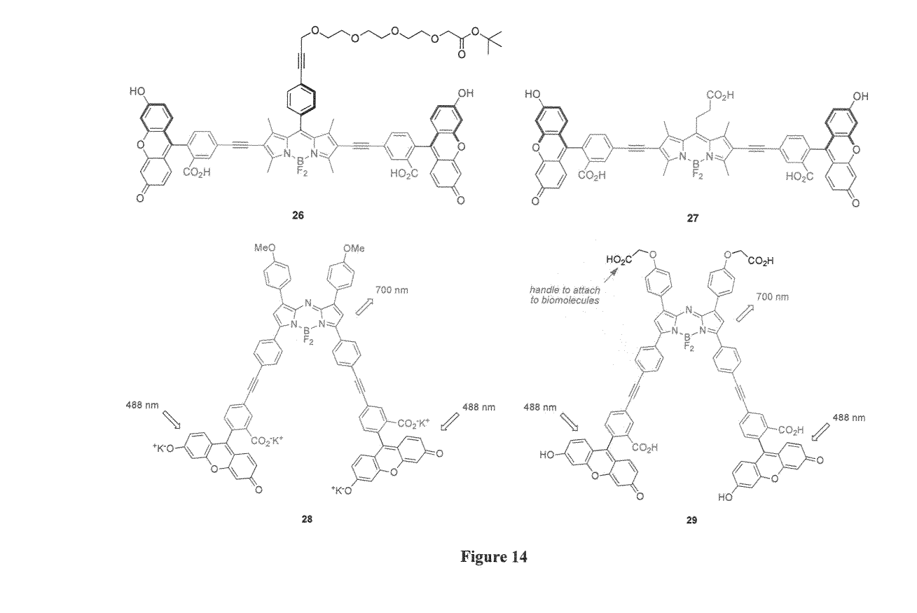

A cassette, in some embodiments, may include a xanthene-BODIPY and/or AzaBODIPY fragments. For example, a cassette may comprise molecules 26-29 (FIG. 14). Other BODIPY and azaBODIPY dyes may be used. Other xanthenes may be used including, for example, halogenated xanthenes (valued for their stabilities at physiological pH, and towards photobleaching) especially the chlorinated xanthenes derived from fragments like 11 and 12. It may also be desirable to include a linker fragment between a donor and an acceptor, particularly ones based on triazole fragments.

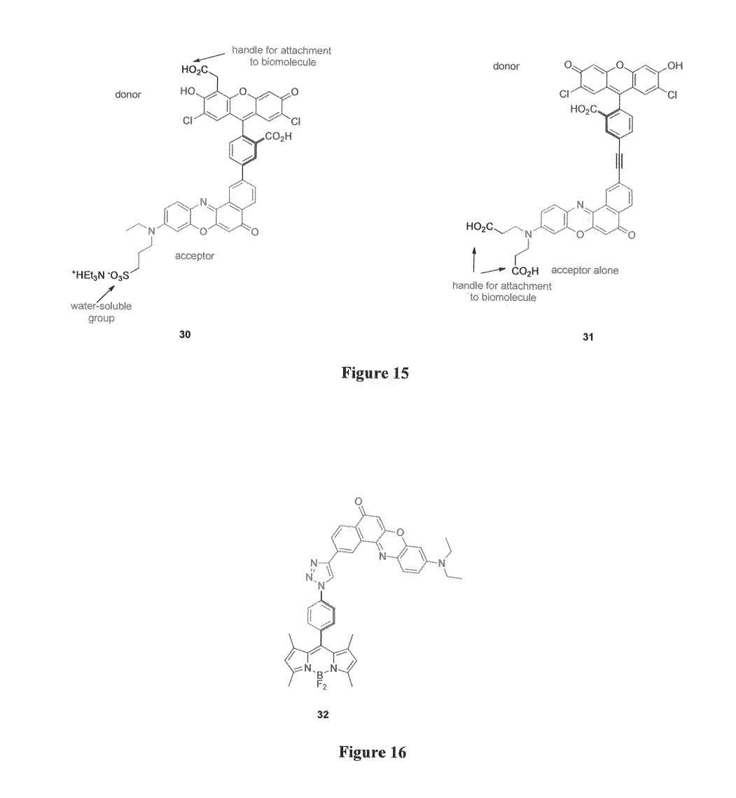

A cassette (e.g., a through-bond energy cassette) may include a xanthene dye, a benzophenoxazine dye, and/or a linker. For example, a cassette may include cassettes 30 and/or 31 (FIG. 15). However other xanthene and benzophenoxazine units may be used. For example, a xanthene-derived fragment may be from fluorescein or other halogenated fluorescein derivatives, and the benzophenoxazine dyes could be derived from Nile Blue based compounds, particularly water soluble compounds related to 17-20.

A cassette (e.g., a through-bond energy cassette) may include a xanthene dye, a BODIPY dye, a benzoxazine dye, and/or a linker. Compound 32 (FIG. 16) is an example of a compound for this (lacking a functional group for attachment to biomolecules).

As will be understood by those skilled in the art who have the benefit of the instant disclosure, other equivalent or alternative through-bond energy compositions, devices, methods, and systems can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. A cassette may or may not comprise, in some embodiments, more than one donor and/or more than one acceptor. In addition, the size of a cassette and/or number of cassettes in a system may be scaled up or down to suit the needs and/or desires of a practitioner. Also, where ranges have been provided, the disclosed endpoints may be treated as exact and/or approximations as desired or demanded by the particular embodiment. In addition, it may be desirable in some embodiments to mix and match range endpoints. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### EXAMPLES

Some specific example embodiments of the disclosure may be illustrated by one or more of the examples provided herein.

### Example 1: Monosulfonation of BODIPY

Sulfonation (e.g., monosulfonation) of BODIPYs may proceed efficiently using 1.2 eq of chlorosulfonic acid (e.g., fresh chlorosulfonic acid) in CH₂Cl₂. The sulfonating agent in CH₂Cl₂ was added dropwise over a few minutes to a solution of the BODIPY starting material at −40° C. After the addition was complete, the cooling bath was removed and the reaction was allowed to warm to 25° C. and stirred for 20 min. For syntheses of compounds like 1a and 2a the reactions were quenched with NaHCO_{3(aq)}, and (after an extraction procedure) the crude products were purified via flash chromatography on silica. A critical observation in this work was the need for the bicarbonate quench; it appears that the protic forms of these sulfonic acids tend to be unstable (though this is not always the case).

Disulfonations of the same starting materials to give products like 1b and 2b feature two equivalents (or slightly more) of the chlorosulfonic acid. Separation in this case is relatively easy because the disulfonic acids precipitate from the dichloromethane solution after 20 min at room temperature. The products were collected by filtration, dissolved in a small amount of aqueous NaHCO₃, evaporated to dryness, then reprecipitated from brine to give essentially pure products. No chromatography is involved, so the procedure may be convenient and amenable to scale up.

A General Procedure for the Preparation of Mono-sulfonated BODIPYs. A solution of chlorosulfonic acid (1.2 eq) in CH₂Cl₂ (dry, 2 ml) was added dropwise to a solution of BODIPY (100 mg) in CH₂Cl₂ (dry, 25 ml) over 10 min under N₂ at −4° C. Then the resulting solution was slowly warmed to room temperature. After 20 min, TLC showed all of the starting material was consumed, then aqueous NaHCO₃ (1.2 eq, 20 ml) was added to neutralize the solution, and the products separated from the CH₂Cl₂ into the aqueous layer. The aqueous layer was evaporated to dryness. The residue was dry-loaded onto a silica gel flash column, and eluted using 15% MeOH/CH₂Cl₂. All the products were isolated as orange powders with an approximate R_{ƒ}=0.4 (20% MeOH/CH₂Cl₂).

Sodium 2-sulfonate-1,3,5,7-tetramethyl-8-(4′-iodophenyl)-4,4-difluoro4-bora-3a, 4a-diaza-s-indacence (1a). 1,3,5,7-Tetramethyl-8-(4′-iodophenyl)-4,4-difluoro-4-bora-3a, 4a-diaza-s-indacence (100 mg, 0.22 mmol) and chlorosulfonic acid (18 μl, 0.27 mmol) were reacted according to the general procedure giving an orange powder (74 mg, 60%). ¹H NMR (500 MHz, CD₃OD) δ 7.95 (d, 2H, J=8.3 Hz), 7.14 (d, 2H, J=8.3 Hz), 6.19 (s, 1H), 2.75 (s, 3H), 2.52 (s, 3H), 1.70 (s, 3H), 1.46 (s, 3H); ¹³C NMR (125 MHz, CD₃OD) δ 159.3, 152.7, 145.7, 142.3, 139.8, 138.8, 134.4, 133.2, 132.7, 130.2, 129.0, 122.8, 94.8, 14.0, 13.6, 13.0, 12.2; MS (ESI) calcd for C₁₉H₁₇BF₂IN₂O₃S⁻ (M—Na)⁻ 529.0066 found 528.8784; IR (thin film) 2922, 1717, 1540, 1312, 1193, 1033, 1006, 678 cm⁻¹.

A General Procedure for the Preparation of Disulfonated BODIPYs. A solution of chlorosulfonic acid (2 eq) in dry CH₂Cl₂ was added dropwise to a solution of BODIPY in dry CH₂Cl₂ over 10 min under N₂ at −40° C. An orange precipitate was formed as the solution mixture warmed slowly to room temperature. The disulfonic acid was isolated by vacuum filtration and treated with water. The aqueous solution was neutralized with NaHCO₃ (2 eq), concentrated to 5-10 ml and treated with brine. The desired product was reprecipitated afterwards to afford an orange solid (85-100% yield).

Disodium 2,6-disulfonate-1,3,5,7-tetramethyl-8-(4′-iodophenyl)-4,4-difluoro-4-bora-3a, 4a-diaza-s-indacence (1b). 1,3,5,7-Tetramethyl-8-(4′-iodophenyl)-4,4-difluoro-4-bora-3a, 4a-diaza-s-indacence (53 mg, 0.118 mmol) and chlorosulfonic acid (16 μl, 0.236 mmol) were reacted according to the general procedure giving an orange powder (68 mg, 88%). ¹H NMR (500 MHz, D₂O) δ 7.84 (d, 2H, J=8.0 Hz), 6.97 (d, 2H, J=8.0 Hz), 2.57 (s, 6H), 1.49 (s, 6H); ¹³C NMR (75 MHz, D₂O) δ 155.5, 145.7, 144.0, 139.2, 133.1, 132.7, 130.6, 129.7, 95.7, 13.7, 13.0; MS (ESI) calcd for C₁₉H₁₇BF₂IN₂O₆S₂⁻ (M−2Na+H)⁻ 608.9634 found 608.9776.

The following documents are hereby incorporated into the instant disclosure by reference:

1. K. Burgess, R. Gibbs, and e. al., (The Texas A&M University System, USA), Through Bond Energy Transfer in Fluorescent Dyes for Labeling Biological Molecules, 2002, patent U.S. Pat. No. 6,340,750 B1.

2. K. Burgess, (The Texas A&M University System, USA). Fluorescent through-bond energy transfer cassettes based on xanthine and pyronin derivatives, 2005, patent U.S. 2005032120.

3. Pyrromethene-BF2 complexes as laser dyes: 1, M. Shah, K. Thangaraj, M. L. Soong, L. Wolford, J. H. Boyer, I. R. Politzer, and T. G. Pavlopoulos, Heteroat. Chem., 1990, 1, 389-99.

4. A novel water-soluble fluorescent probe: Synthesis, luminescence and biological properties of the sodium salt of the 4-sulfonato-3,3′,5,5′-tetramethyl-2,2′-pyrromethen-1,1′-BF2 complex, H. J. Wories, J. H. Koek, G. Lodder, J. Lugtenburg, R. Fokkens, 0. Driessen, and G. R. Mohn, Recl. Trav. Chim. Pays-Bas, 1985, 104,288-91.

5. Water soluble distyryl-boradiazaindacenes as efficient photosensitizers for photodynamic therapy, S. Atilgan, Z. Ekmekci, A. L. Dogan, D. Guc, and U. Akkaya Engin, Chem. Commun., 2006, 4398-400.

6. 4,4-Difluoro-4-bora-3a,4a-diaza-s-indacene (BODIPY) dyes modified for extended conjugation and restricted bond rotations, J. Chen, A. Burghart, A. Derecskei-Kovacs, and K. Burgess, J. Org. Chem., 2000, 65, 2900-6.

7. Synthesis of Fluorinated Fluoresceins, W.-C. Sun, K. R. Gee, D. H. Klaubert, and R. P. Haugland, J. Org. Chem, 1997, 62, 6469-75.

8. D. H. Klaubert, and K. R. Gee, Molecular Probes, Inc., Synthesis of Fluorinated Xanthene Derivatives, 2001, U.S. Pat. No. 6,229,055 B1.

9. K. R. Gee, M. Poot, D. H. Klaubert, W.-C. Sun, R. P. Haugland, and F. Mao, Molecular Probes, Inc., Fluorinated Xanthene Derivatives, 2000, U.S. Pat. No. 6,162,931.

10. Improvement and Biological Applications of Fluorescent Probes for Zinc, ZnAFs, T. Hirano, K. Kikuchi, Y. Urano, and T. Nagano, J. Am. Chem Soc., 2002, 124, 6555-62.

11. Xanthenes: fluorone derivatives. 1, J. Shi, X. Zhang, and D. C. Neckers, J. Org. Chem., 1992, 57, 4418-21.

12. Effect of Binding and Conformation on Fluorescence Quenching in New 2′-7′-Dichlorofluorescein Derivatives., B. A. Sparano, S. P. Shahi, and K. Koide, Org. Lett., 2004, 6, 1947-9.

13. Improved Synthetic Procedures for 4,7,2′,7′-Tetrachloro- and 4′,5′-Dichloro-2′,7′-dimethoxy-5(and 6)-carboxyfluoresceins, M. H. Lyttle, T. G. Carter, and R. M. Cook, Org. Process Res. Dev., 2001, 5, 45-9.

14. A general method for the covalent labeling of fusion proteins with small molecules in vivo, A. Keppler, S. Gendreizig, T. Gronemeyer, H. Pick, H. Vogel, and K. Johnsson, Nat. Biotechnol., 2003, 21, 86-9.

15. U.S. application Ser. No. 10/876,919, published as US2005/0032120.

16. U.S. Pat. No. 6,340,750 issued to Burgess et al. on Jan. 22, 2002

What is claimed is:

1. An energy transfer cassette comprising a through-bond energy transfer cassette comprising a donor and an acceptor, wherein at least one of the donor and acceptor comprises a 4,4-difluoro-4-bora-3a,4a-diaza-s-indacene (BODIPY), a sulfonated BODIPY, a rosamine, a rhodamine, dichlorofluorescein, a Nile Red, a Nile Blue, a benzophenoxazine, an AzoBODIPY, Xanthene-AzoBODIPY, a xanthene-benzophenoxazine, benzoxazine, or fluoroscein.

2. An energy transfer cassette according to claim 1, further comprising a linker covalently linked to the donor and the acceptor.

3. An energy transfer cassette according to claim 1, wherein at least one of the donor and the acceptor comprises a dye selected from the group consisting of dyes 1-5, dyes 6a-6h, dye 6h-strep, dyes 8a-8o, dyes 9a-9f, and dyes 10-20.

4. An energy transfer cassette according to claim 1, further comprising the donor operable to absorb electromagnetic energy at a first wavelength and the acceptor operable to emit electromagnetic energy at a second wavelength, wherein the second wavelength is longer than the first wavelength.

5. An energy transfer cassette according to claim 1, wherein the donor π-system and acceptor π-system are not π-conjugated in a ground state due to the presence of one or more twists that render the donor π-system and acceptor π-system non-planar.

6. An energy transfer cassette according to claim 1, wherein the energy transfer cassette is selected from the group consisting of cassettes 21-33.

7. An energy transfer cassette according to claim 1, wherein the energy transfer cassette is water soluble.

8. An energy transfer cassette according to claim 1, further comprising a molecule selected from the group consisting of a protein, a nucleic acid, a carbohydrate, a lipid, and combinations thereof.

9. An energy transfer cassette according to claim 1, wherein at least one of the donor and acceptor comprises a benzophenoxazine.

10. An energy transfer cassette according to claim 9, further comprising a dendric wedge configured and arranged to shield the benzophenoxazine.

11. A method for sulfonating a 4,4-difluoro-4-bora-3a,4a-diaza-s-indacene comprising:
contacting chlorosulfonic acid and 4,4-difluoro-4-bora-3a,4a-diaza-s-indacene in an organic solvent at a temperature of from about −45° C. to about −20° C. to form a mixture;
warming the mixture to about room temperature; and
contacting the mixture with a base,
wherein a sulfonated 4,4-difluoro-4-bora-3a,4a-diaza-s-indacene is formed.

12. A method according to claim 11, wherein the organic solvent comprises dichloromethane.

13. A method according to claim 11, wherein the concentration of chlorosulfonic acid is about 1.2 eq.

14. A method according to claim 11, wherein the base comprises bicarbonate.

15. A fluorescent dye comprising a molecule selected from the group consisting of dye 1, dye 2, dye 3, dye 4, dye 5, dye 6, dye 9, dye 10, dye 11, dye 12, dye 13, dye 14, dye 15, dye 16, dye 17, dye 18, dye 19, and dye 20.

16. A fluorescent dye according to claim 14, wherein the fluorescent dye is water soluble.

17. A system for detecting a molecule, the system comprising:
a through-bond energy transfer cassette comprising
a donor and an acceptor, wherein at least one of the donor and acceptor comprises a 4,4-difluoro-4-bora-3a,4a-diaza-s-indacene (BODIPY), a sulfonated BODIPY, a rosamine, a rhodamine, dichlorofluorescein, a Nile Red, a Nile Blue, a benzophenoxazine, an AzoBODIPY, Xanthene-AzoBODIPY, a xanthene-benzophenoxazine, benzoxazine, or fluoroscein and the donor and the acceptor are non-planar relative to each other in a ground state;
a linker covalently linked to the donor and the acceptor; and
a functional group linked to at least the donor, the acceptor, or the linker.

18. A system according to claim 17 further comprising a second through-bond energy transfer cassette comprising
a donor and an acceptor, wherein at least one of the donor and acceptor comprises a 4,4-difluoro-4-bora-3a,4a-diaza-s-indacene (BODIPY), a sulfonated BODIPY, a rosamine, a rhodamine, dichlorofluorescein, a Nile Red, a Nile Blue, a benzophenoxazine, an AzoBODIPY, Xanthene-AzoBODIPY, a xanthene-benzophenoxazine, benzoxazine, or fluoroscein;
a linker covalently linked to the donor and the acceptor; and
a functional group linked to at least the donor, the acceptor, or the linker.

19. A system according to claim 18, wherein the fluorescence profile of the first through-bond energy transfer cassette is distinguishable from the fluorescence profile of the second through-bond energy transfer cassette.

20. A system according to claim 18, wherein the first donor and the second donor are the same.
